(19) United States
(12) Patent Application Publication
(10) Pub. No.: US 2022/0036468 A1
(43) Pub. Date: Feb. 3, 2022

(54) DISCREPANCY AUDIT SYSTEM AND METHOD FOR USE THEREWITH

(71) Applicant: Enrollease, Inc., San Francisco, CA (US)

(72) Inventors: Courtney Guertin, San Francisco, CA (US); David Reid, Las Vegas, NV (US)

(73) Assignee: Enrollease, Inc., San Francisco, CA (US)

(21) Appl. No.: 16/944,833

(22) Filed: Jul. 31, 2020

(51) Int. Cl. G06Q 40/08; G16H 10/60

(52) U.S. Cl. CPC G06Q 40/08; G16H 10/60

(57) ABSTRACT

A discrepancy audit system operates by generating discrepancy audit data indicating instances of the partner benefits records that fail to match corresponding instances of the system benefits records and generating an interactive interface for display via a display device. The interactive interface displays the discrepancy audit data by displaying the instances of the partner benefits records contemporaneously with the corresponding instances of the system benefits records. In response to a user selection of a first instance of the instances of the partner benefits records, indicating the first instance of the instances of the partner benefits records is correct, automatically indicating a corresponding first instance of the corresponding instances of the system benefits records is incorrect, and generating corrected system benefits records indicating the first instance of the instances of the partner benefits records. In response to a user selection of a second instance of the corresponding instances of the system benefits records, indicating the second instance of the corresponding instances of the system benefits records is correct, and automatically indicating a corresponding second instance of the instances of the partner benefits records is incorrect.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISC

Not applicable.

### BACKGROUND

### Technical Field

This invention relates generally to automated administrative processing systems used in conjunction with client/server and other network architectures for managing employee benefits and other human resource functions.

### Description of Related Art

[symbol]

### BRIEF DESCRIPTION OF THE SEVERAL VIEWS OF THE DRAWING(S)

FIG. 1 is a schematic block diagram of an embodiment of an admin processing system;

FIG. 2A is a schematic block diagram of a client device in accordance with various embodiments;

FIG. 2B is a schematic block diagram of one or more admin subsystems in accordance with various embodiments;

FIG. 3 is a schematic block diagram of a discrepancy audit system in accordance with various embodiments;

FIGS. 4A-4W are screen displays of an interactive interface in accordance with various embodiments; and

FIG. 5 is a flowchart representation of a method in accordance with various embodiments.

### DETAILED DESCRIPTION

One or more embodiments are now described with reference to the drawings, wherein like reference numerals are used to refer to like elements throughout. In the following description, for purposes of explanation, numerous details are set forth in order to provide a thorough understanding of the various embodiments. It is evident, however, that the various embodiments can be practiced without these details and without applying to any particular networked environment or standard.

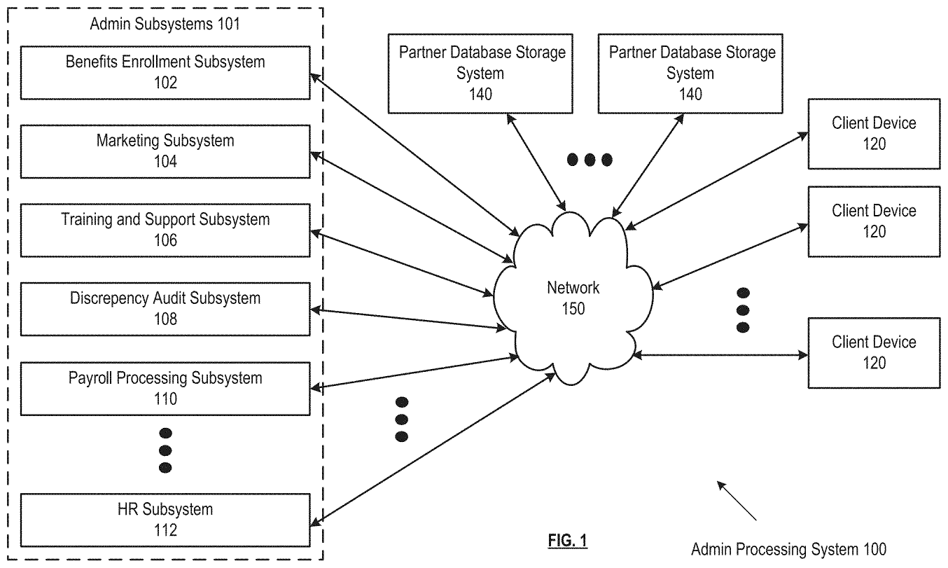

FIG. 1 presents an admin (administrative) processing system 100, which can include one or more admin subsystems 101 that communicate bidirectionally with one or more client devices 120 via a wired and/or wireless network 150. The admin subsystems 101 can include, for example, a benefits enrollment subsystem 102, a marketing subsystem 104, a training and support subsystem 106, a discrepancy audit subsystem 108, a payroll processing subsystem 110, HR subsystem and/or other admin subsystems for providing automated processing support to one or more partners, such as partner companies. Some or all of the subsystems 101 can utilize the same processing devices, memory devices, and/or network interfaces, for example, running on a same set of cloud servers or other shared servers connected to network 150. Alternatively or in addition, some or all of the subsystems 101 be assigned their own processing devices, memory devices, and/or network interfaces, for example, running separately on different sets of servers connected to network 150. Some or all of the subsystems 101 can interact directly with each other, for example, where one subsystem's output is transmitted directly as input to another subsystem via network 150. Network 150 can include one or more wireless, optical and/or wired communication systems; one or more non-public intranet systems and/or public Internet systems; and/or one or more local area networks (LAN) and/or wide area networks (WAN).

The admin processing system 100 can further include one or more partner database storage systems 140 corresponding to, for example, one or more partner companies. The partner database storage systems 140 can include one or more servers, one or more memory devices of one or more subsystems 101, and/or one or more other memory devices connected to network 150. Each partner database storage system 140 can store one or more shared databases and/or one or more files stored on one or more memory devices that include records and other database entries as described herein. The shared databases and/or files can each be utilized by some or all of the subsystems of the admin processing system 100, allowing some or all of the subsystems 101 and/or client devices 120 to retrieve, review, process, edit, add, or delete entries to the one or more databases and/or files.

One or more client devices 120 can each be associated with one or more users of one or more subsystems 101 of the admin processing system 100. Some or all of the client devices 120 can be associated with insurance companies, brokers, benefits administrators, systems administrators, as well as human resources (HR) professionals, employees, or other individual users for example, located at a partner company or other location providing services to one or more partner companies. Some of the client devices 120 can correspond to one or more administrators of one or more subsystems of the admin processing system 100, allowing administrators and other professionals to operate, manage, supervise, or otherwise support the functions of one or more subsystems 101 for which they are authorized to access.

Some or all of the subsystems 101 of the admin processing system 100 can include a server that presents a website for operation via a browser of client devices 120. Alternatively or in addition, each client device 120 can store specific application data, a corresponding database and/or other data in a memory, data corresponding to some or all subsystems 101, for example, a subset of the subsystems 101 that are relevant to the user of the client device 120. A processor of the client device 120 can operate via a display device to display an interactive interface based on instructions in the data stored in memory and/or received via the network 150. For example, a website implemented by a subsystem 101 can operate via the application. Some or all of the websites presented can correspond to multiple subsystems 101, for example, where the multiple subsystems share the server presenting the website.

Each subsystem 101 can be configured for user authentication such as two factor authentication or other authentication to prohibit unauthorized access. In addition, the admin subsystems 101 and partner database storage systems 140 can employ encryption, such as AES256 and/or other encryption algorithm to protect the security of data stored therein. Furthermore, the network 150 can be configured for secure communications between the admin subsystems 101, the client devices 120 and the database storage system 140 to protect the security of data communicated between the admin subsystems 101, the client devices 120 and the database storage system 140.

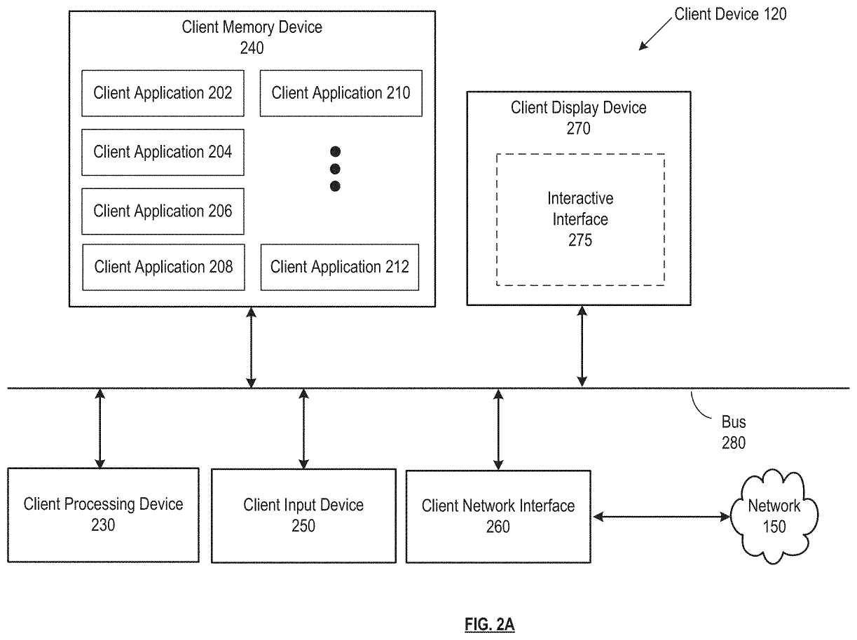

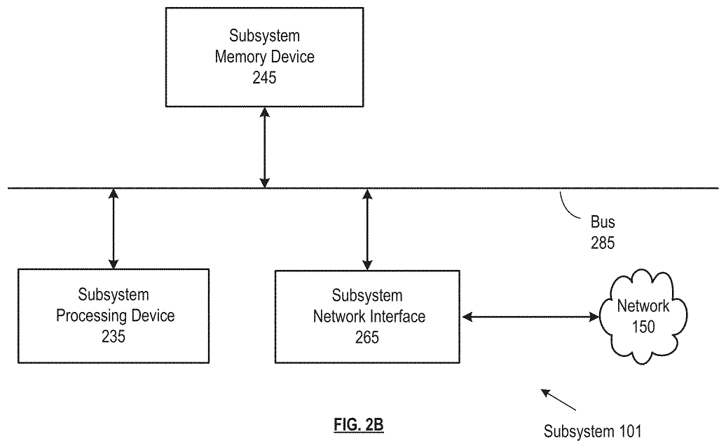

FIG. 2A presents an embodiment of client device 120. Each client device 120 can include one or more client processing devices 230 that each include a processing circuit, one or more client memory devices 240, one or more client input devices 250, one or more client network interfaces 260 operable to more support one or more communication links via the network 150 indirectly and/or directly, and/or one or more client display devices 270, connected via bus 280. While a particular bus structure is shown for purposes of illustration in a block diagram, other structures including multiple buses and/or direct connections between functional blocks. Client applications 202, 204, 206, 208, 210 and/or 212 can correspond to subsystems 102, 104, 106, 108, 110 and/or 112 of the medical scan processing system respectfully. Each client device 120 can receive the application data from the corresponding subsystem 101 via network 150 by utilizing network interface 260, for storage in the one or more memory devices 240. In various embodiments, some or all client devices 120 can include a computing device associated with a user of one or more subsystems 101 as described herein.

In various embodiments, the memory device 240 can store executable instructions that, when executed by the processing device 230, facilitate the performance of operations by the client device 120, as discussed herein. In particular, the one or more processing devices 230 can generate an interactive interface 275 on the one or more client display devices 270 in accordance with one or more of the client applications 202, 204, 206, 208, 210, and/or 212, for example, where the same or different interactive interface 275 is displayed for some or all of the client applications in accordance with the website presented by the corresponding subsystem 102, 104, 106, 108, 110 and/or 112. The user can provide input in response to menu data, selectable links and/or other prompts presented by the interactive interface via the one or more client input devices 250, which can include a microphone, mouse, keyboard, touchscreen of display device 270 itself or other touchscreen, and/or other device allowing the user to interact with the interactive interface 275. The one or more processing devices 230 can process data and/or send raw or processed data to the corresponding subsystem 101, and/or can receive and/or generate new data in response for presentation via the interactive interface 275 accordingly, by utilizing network interface 260 to communicate bidirectionally with one or more subsystems 101, partner database storage systems 140 and/or or other systems via the network 150.

FIG. 2B presents an embodiment of a subsystem 101, which can be utilized in conjunction with subsystem 102, 104, 106, 108, 110 and/or 112. Each subsystem 101 can include one or more subsystem processing devices 235 that each include a processing circuit, one or more subsystem memory devices 245, and/or one or more subsystem network interfaces 265, connected via bus 285. While a particular bus structure is shown for purposes of illustration in a block diagram, other structures including multiple buses and/or direct connections between functional blocks. The subsystem memory devices 245 can store executable instructions that, when executed by the one or more subsystem processing devices 235, facilitate performance of operations by the subsystem 101, as described for each subsystem herein.

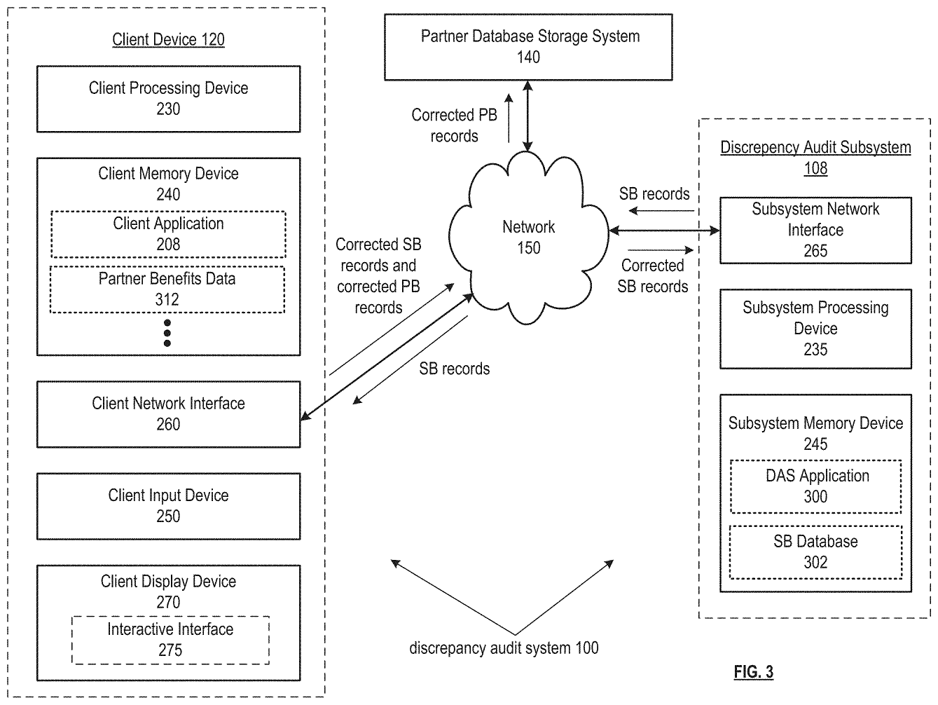

FIG. 3 is a schematic block diagram of a discrepancy audit system in accordance with various embodiments. In particular, a discrepancy audit system 100 is presented that includes a discrepancy audit subsystem 108 and client device 120. The partner database storage system 140 maintains a database of partner benefit (PB) records. The subsystem memory device 245 of discrepancy audit subsystem 108 includes a discrepancy audit system application 300 and system benefits database 302 that stores system benefits (SB) records corresponding, for example, up to date system-maintained copies of the partner benefits records of partner database storage system 140 and other partner database storage systems 140 corresponding to, for example, other partners.

The client device 120 executes the client application 208, such as a browser, operating system or other general purpose application, a database system and/or a special purpose client application. The discrepancy audit subsystem 108 executes the discrepancy audit subsystem (DAS) application 300 such as an operating system or other general purpose application along with a database system, a special purpose server application or other application. The client application 208 and DAS application 300 of the discrepancy audit system 100 allow that client device 120 and the discrepancy audit subsystem 108 to cooperate, permitting a user of the client device 120 to perform the various functions of the discrepancy audit system 100. It should be noted that this cooperation can include functions solely performed by the client device 120, functions performed solely by the discrepancy audit subsystem 108 and functions performed jointly via both devices. These functions include the various functions of a discrepancy audit of benefit records corresponding to one or more partners whose benefits are managed by the user.

In various embodiments, the discrepancy audit functions performed by the discrepancy audit system 100 can include the following.

(a) obtaining partner benefits records. These partner benefits records can, for example, correspond to records from a random audit, systematic audit, scheduled audit, other type of audit, new records, recent data, other updates and/or other benefits records of a particular partner. These partner benefits records can be stored locally as the partner benefits data 312 and/or obtained from another source via network 150, such as the partner database storage system 140.

(b) Comparing the partner benefits records to system benefits records from the system benefits database, for example, to determine whether or not here are instances in the partner benefits records that fail to match corresponding instances of the system benefits records indicating either an error present in the systems benefits records, an error in the partner benefits records, or updated data reflected in the partner benefits records requiring a corresponding update in the systems benefits records, e.g. a change of name, address, change of benefits, change in dependents, etc. In various embodiments, the system benefits records and partner benefits records contain the same fields of information corresponding to, for example, a name, a designation as an employee, spouse, child or other dependent, gender, address, social security number, identification number, office address, employee type, employee status, hire date, birth date, termination date, department, division, job classification, job title, medical insurance plan data, life insurance plan data, dental insurance plan data, disability insurance plan data, accidental death and dismemberment plan data, etc.

(c) Generating discrepancy audit data that indicates the instances of the partner benefits records that fail to match corresponding instances of the system benefits records. The discrepancy audit data can include instances, such as content of the specific fields of information where the partner benefits records do not match the system benefits records. The discrepancy audit data can further include: an audit time, an audit date, a number of the instances of the partner benefits records that fail to match the corresponding instances of the system benefits records, a number of different employees reflected in the instances of the partner benefits records that fail to match the corresponding instances of the system benefits records, a number of dependents reflected in the instances of the partner benefits records that fail to match the corresponding instances of the system benefits records, and or other audit data.

(d) Generating an interactive interface 275 for display via a display device 270. The interactive interface 275 displays the discrepancy audit data by displaying the instances of the partner benefits records contemporaneously with the corresponding instances of the system benefits records. In particular, the interactive interface 275 permits the user to review the various discrepancies and correct the system benefits data or the partner benefits data or to otherwise generate a consensus.

(e) In response to a user selection via the interactive interface 275, of a first instance of the instances of the partner benefits records, the interactive interface 275 responds by providing an indication that the first instance of the instances of the partner benefits records is correct, by automatically providing an indication that a corresponding first instance of the corresponding instances of the system benefits records is incorrect. This selection can be as simple as a single click of the mouse or other pointing device that not only allows the user to select the correct information, but also to automatically—and without any additional user intervention—select the information that that is incorrect. The discrepancy audit subsystem 108 also responds by generating corrected system benefits records indicating the first instance of the instances of the partner benefits records. In this fashion, corrections data can be generated to facilitate the correction of the incorrect portion of the system benefits records to match the corresponding portion of the partner benefits records. In various embodiments, indicating the first instance of the instances of the partner benefits records is correct includes a first visual indication via the interactive interface, and automatically indicating the corresponding first instance of the instances of the partner benefits records is incorrect includes a second visual indication via the interactive interface. These visual indications can be changes in text formatting, such as underlining, strike-through, italics, bold, and/or text color, changes in background color, pop-up notifications, or other indications.

(f) In response to a user selection, via the interactive interface, of a second instance of the corresponding instances of the system benefits records, indicating the second instance of the corresponding instances of the system benefits records is correct, and automatically indicating a corresponding second instance of the instances of the partner benefits records is incorrect. Again, this selection can be as simple as a single click of the mouse or other pointing device that not only allows the user to select the correct information, but also to automatically—and without any additional user intervention—select the information that that is incorrect. The discrepancy audit subsystem 108 can also respond by generating corrected partner benefits records indicating the second instance of the corresponding instances of the system benefits records. In this fashion, corrections data can be generated to facilitate the correction of the incorrect portion of the partner benefits records to match the corresponding portion of the systems benefits records. In various embodiments, indicating the second instance of the corresponding instances of the system benefits records is correct includes a third visual indication via the interactive interface, and automatically indicating the corresponding second instance of the instances of the partner benefits records is incorrect includes a fourth visual indication via the interactive interface. These visual indications can be changes in text formatting, such as underlining, strike-through, italics, bold, and/or text color, changes in background color, pop-up notifications, or other indications.

(g) Facilitating updates to the system benefits records based on the corrected system benefits records. In various embodiments, system benefits records are obtained from the system benefits database 302 and facilitating updates to the system benefits records includes importing the corrected system benefits records to the system benefits database 302.

(h) Facilitating updates to the partner benefits records. In various embodiments, the corrected partner benefits records can be pushed to the partner database storage system 140, pulled by the partner database storage system 140 in realtime, as part of a periodic update or other exporting of the corrected partner benefits records to the partner database storage system 140.

In various embodiments, the discrepancy audit system 100 can perform other discrepancy audit functions as well. For example, obtaining partner benefits records in (a) above, can include importing a partner benefits record file. The discrepancy audit system 100 can further operate by: processing the partner benefits record file based on a plurality of warning or error conditions; generating one or more warning or error notifications in response to the processing; and presenting the one or more warning or error notifications for display via the interactive interface. In another example, the interactive interface 275 can respond to a user selection to display the contents of the partner benefits record file and edit the partner records file to correct any warnings or errors. In a further example, the interactive interface 275 can respond to a user selection to present details corresponding to a selected one of the instances of either the systems benefits records or the partner benefits records by presenting the details corresponding to the selected one of the instances of the systems benefits records or the partner benefits records for display.

In various embodiments, the discrepancy audit system 100 can operate based on a set of rules to automatically determine whether particular instances of the systems benefits records or the partner benefits records appear to be correct or incorrect. For example, the discrepancy audit system 100 can further operate by: processing the instances of the partner benefits records and the corresponding instances of the system benefits records based on a set of rules; indicating, via the interactive interface 275 and based on the processing, a first subset of the instances of the partner benefits records is correct and a corresponding first subset of the corresponding instances of the system benefits records is incorrect; and indicating, via the interactive interface and based on the processing, a second subset of the instances of the partner benefits records is incorrect and a corresponding second subset of the corresponding instances of the system benefits records is correct. These correct/incorrect indications can also be changes in text formatting, such as underlining, strike-through, italics, bold, and/or text color, changes in background color, pop-up notifications, or other indications.

The set of rules can include one or more rules of inconsistency. If an instance of a partner benefits record is determined to be inconsistent when compared, for example, with other instances of the same record and further, the corresponding systems record is determined to be not inconsistent (i.e. consistent) then the instance of the partner benefits record can be deemed incorrect and the corresponding instance of the system benefits record can be deemed correct—and vice versa. Examples of inconsistency rules include, a termination date that falls before the hire date, a birthdate of an employee that would yield an age less than the legal minimum for employment, etc. Other rules can compare field values with a list of acceptable values that, for example, may or may not be customized for a particular partner. Such rules can determine when a deductible amount or benefits limit does not correspond to a valid plan. Job titles can be compared to a list of acceptable titles for a particular partner. Errors can be determined based on a comparison to other sources of information. For example, an address can be deemed incorrect that fails to correspond to a valid post office address. Fields for city, state and county can be compared to standard geographical information to determine inconsistencies, correctness and/or apparent errors, for example, when a zip code, city and/or state do not match one another The set of rules can include formatting rules, for example, where a field cannot contain a numerical character or must contain a valid number. Various fields can be compared to spelling lists to detect instances with misspellings. Instances that have previously been corrected, or determined to be correct via previous manual selection by a user can be presumed to be correct if future discrepancies occur. Other rules can also be employed in the set of rules as needed.

When the set of rules is applied and the first and second subsets are identified, the automatic determinations are indicated in the interactive interface 275 and can be reviewed and reversed, if necessary in response to actions of the user. For example, when the first instance of the instances of the partner benefits records is a member of the second subset, the user selection, via the interactive interface, of the first instance of the instances of the partner benefits records reverses an indication that the first instance of the instances of the partner benefits records is incorrect. In this fashion, a rules-based automatic indication that an instance a partner benefits record incorrect can be reversed and indicated to be correct. Similarly, when the second instance of the corresponding instances of the system benefits records is a member of the first subset, the user selection via the interactive interface, of the second instance of the corresponding instances of the system benefits records reverses an indication that the second instance of the corresponding instances of the system benefits records is incorrect. In this fashion, a rules-based automatic indication that an instance a system benefits record incorrect can be reversed and indicated to be correct.

In some cases, the rules may not be able to resolve a discrepancy. In this case, the discrepancy audit system can operate by indicating, via the interactive interface, that a third subset of the instances of the partner benefits records and a corresponding third subset of the corresponding instances of the system benefits records cannot be determined to be either correct or incorrect. These indications can also be changes in text formatting, such as underlining, strike-through, italics, bold, and/or text color, changes in background color, pop-up notifications, or other indications, or by contrasting with other automatic indications by not making changes to the text formatting, background color, etc. It should be noted that the first, second and third subsets are mutually non-intersecting and together span the non-matching instances of the discrepancy audit data.

It should also be noted that, while the foregoing has described an interactive review procedure where instances of correct benefits records are indicated by the user via the interactive interface 275 and incorrect instances are automatically flagged, conversely, a similar procedure could be implemented where instances of incorrect benefits records are indicated by the user and correct instances are automatically flagged. Furthermore, the interactive interface 275 can include a button, menu option or other selection mechanism that allows a user to select whether correct or incorrect instances are selected.

Further examples including many optional functions and figures are described in conjunction with the Figures that follow.

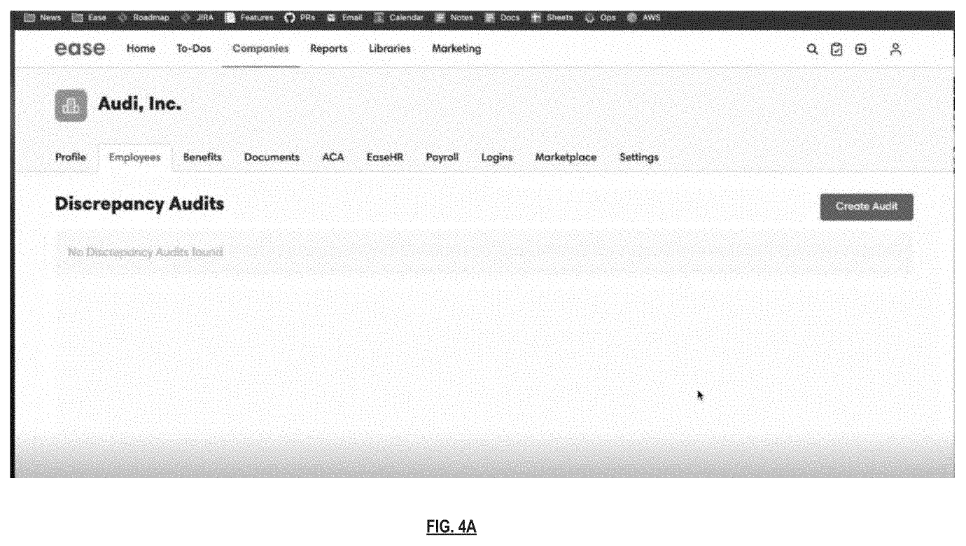

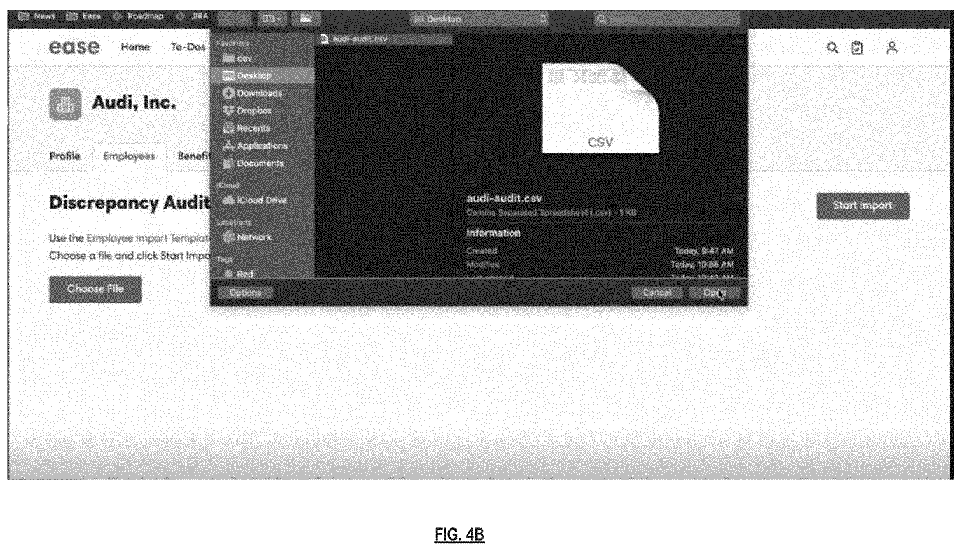

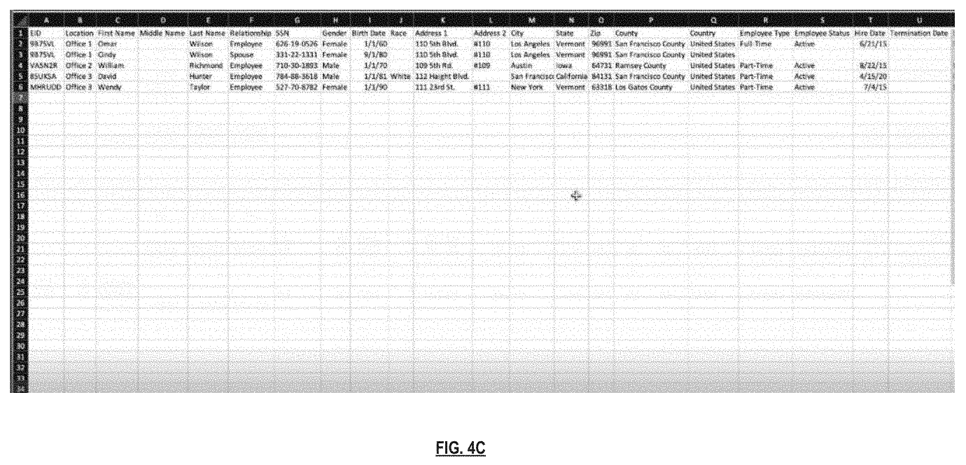

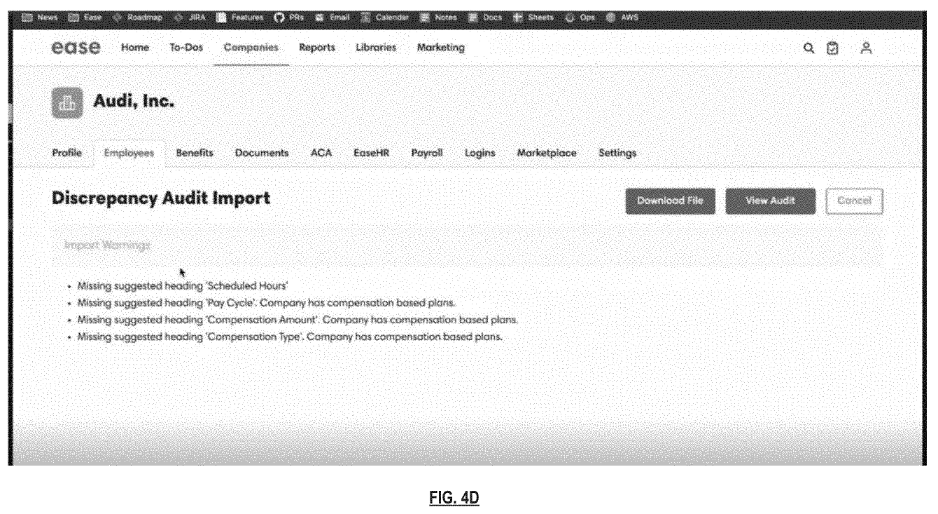

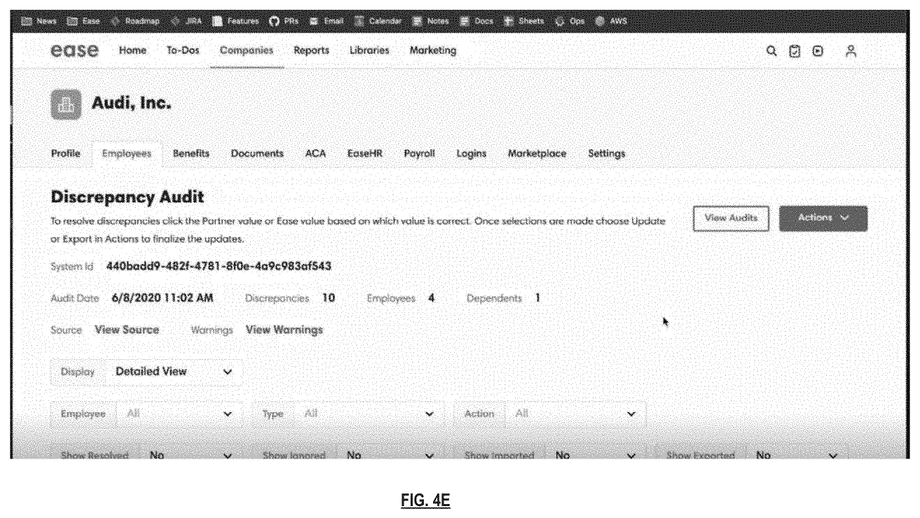

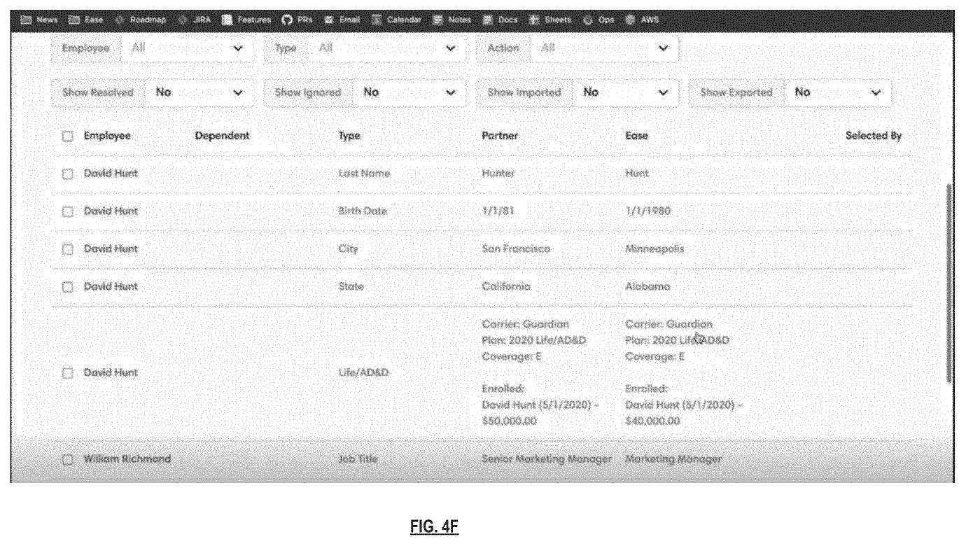

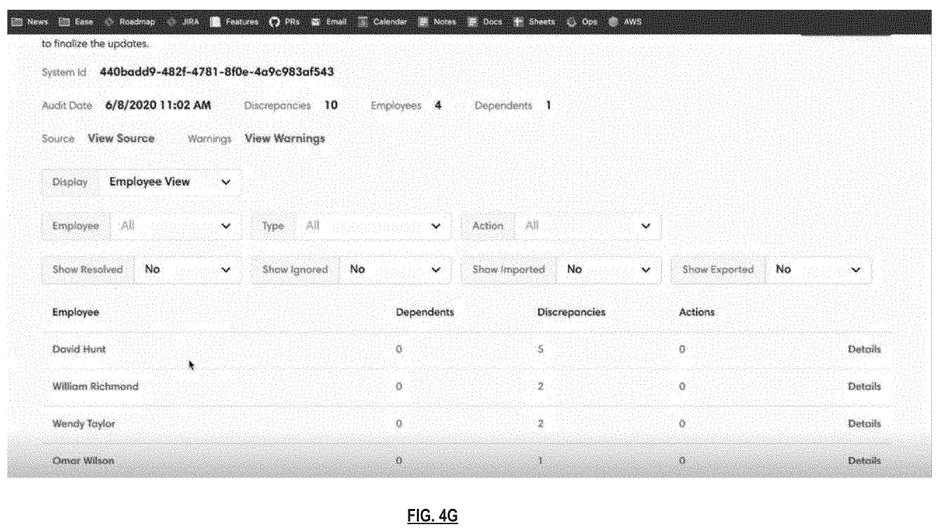

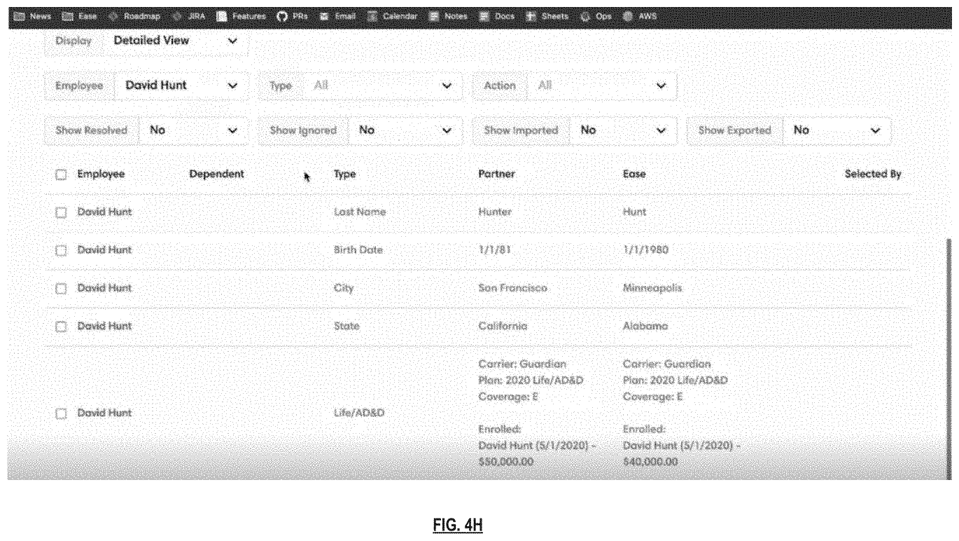

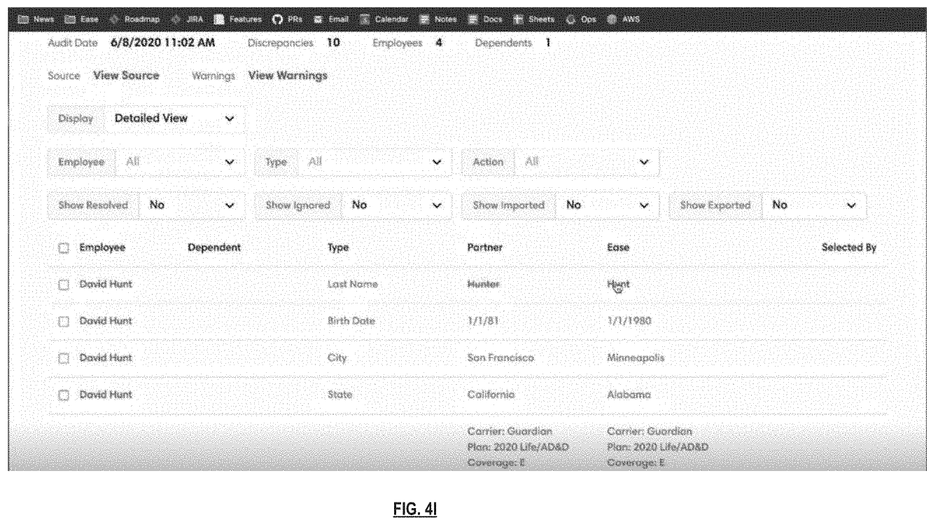

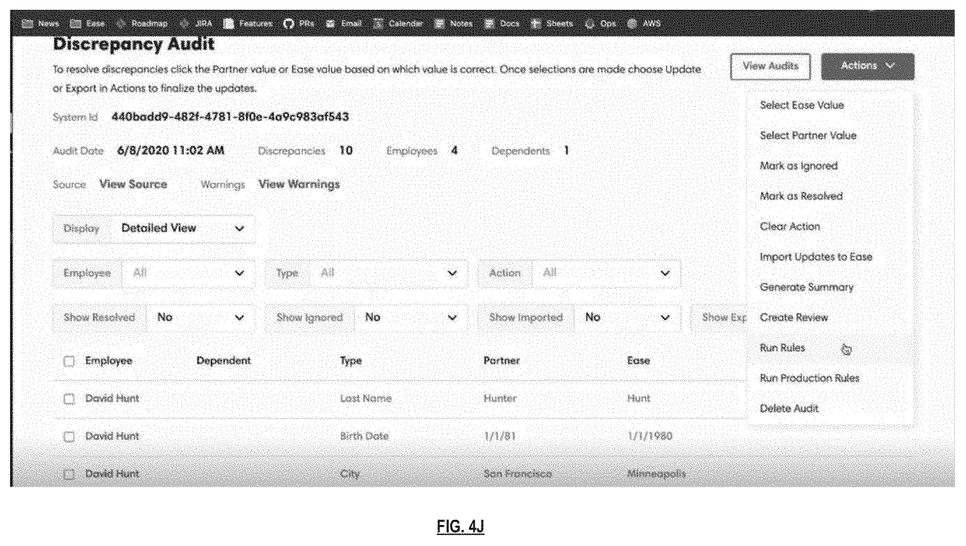

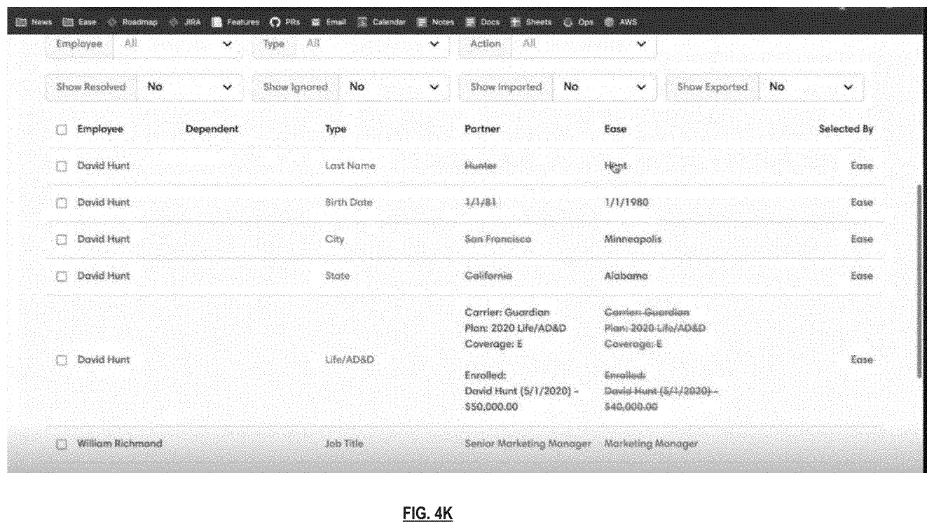

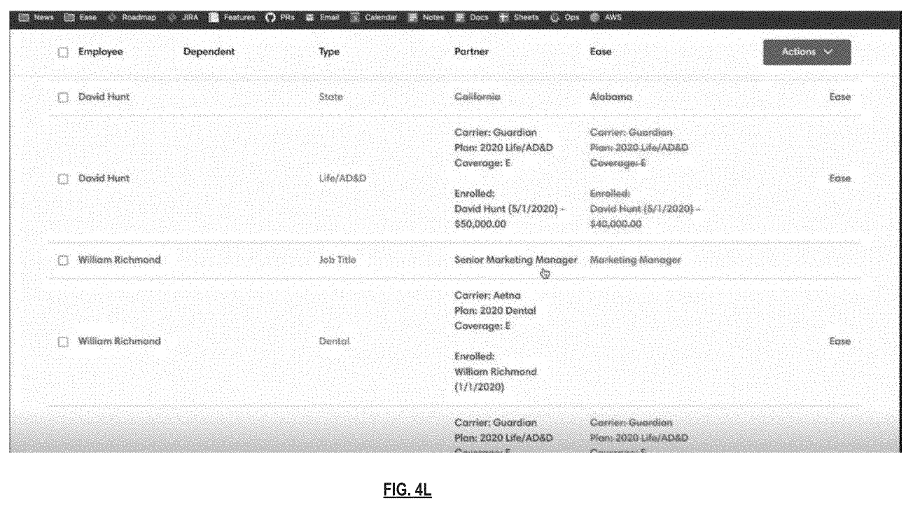

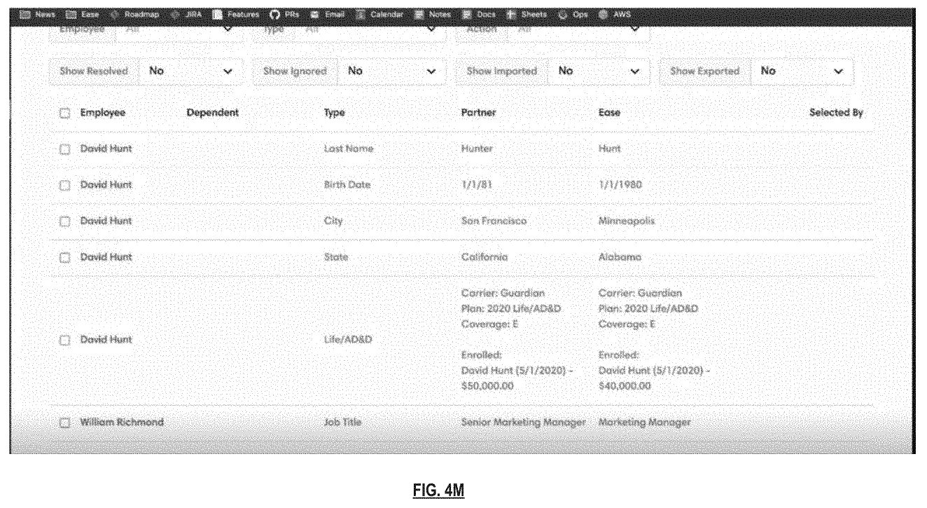

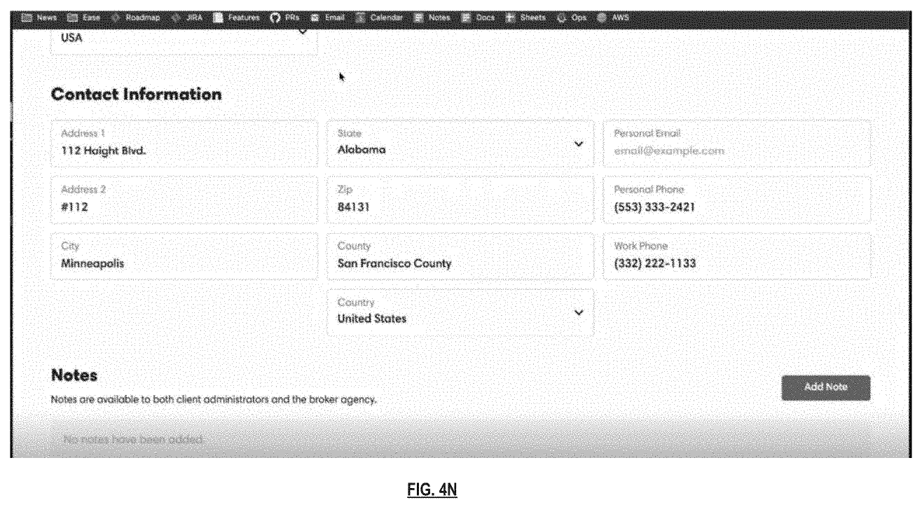

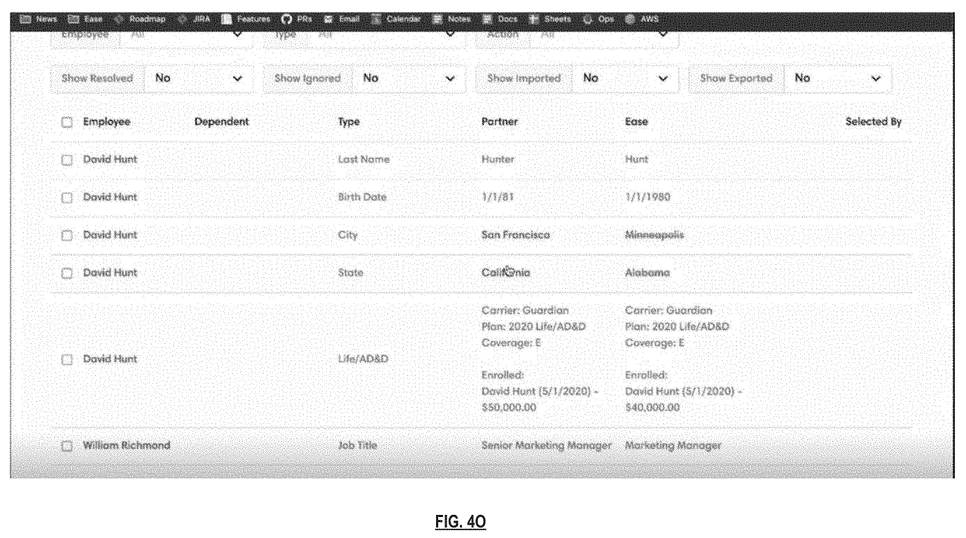

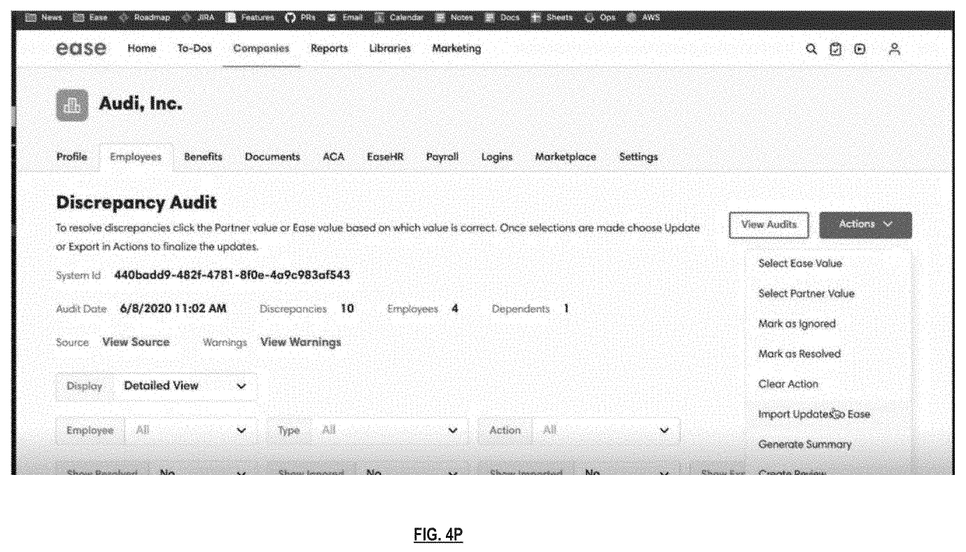

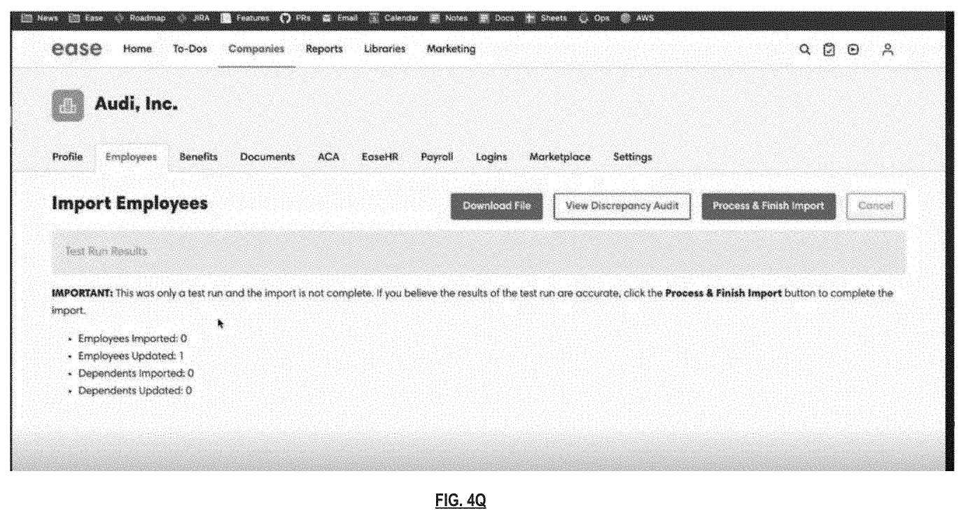

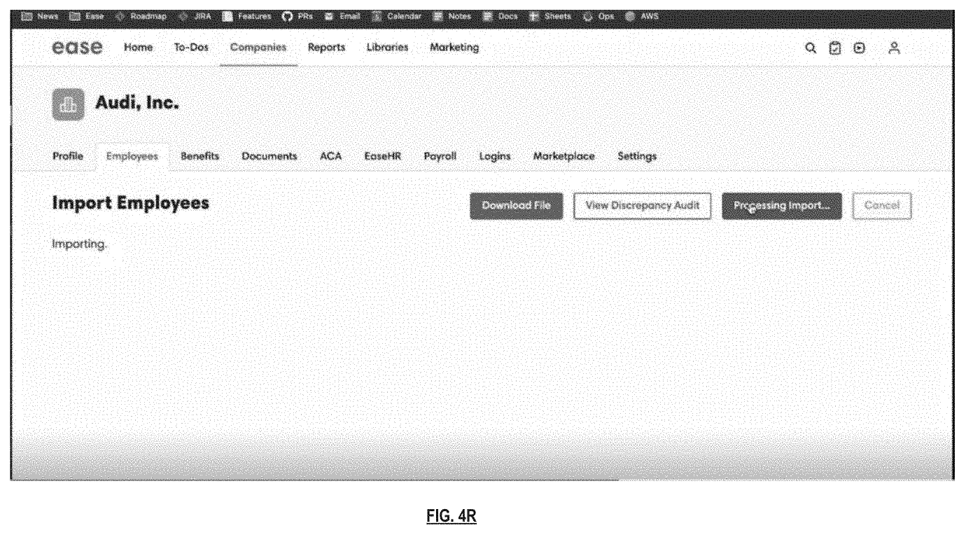

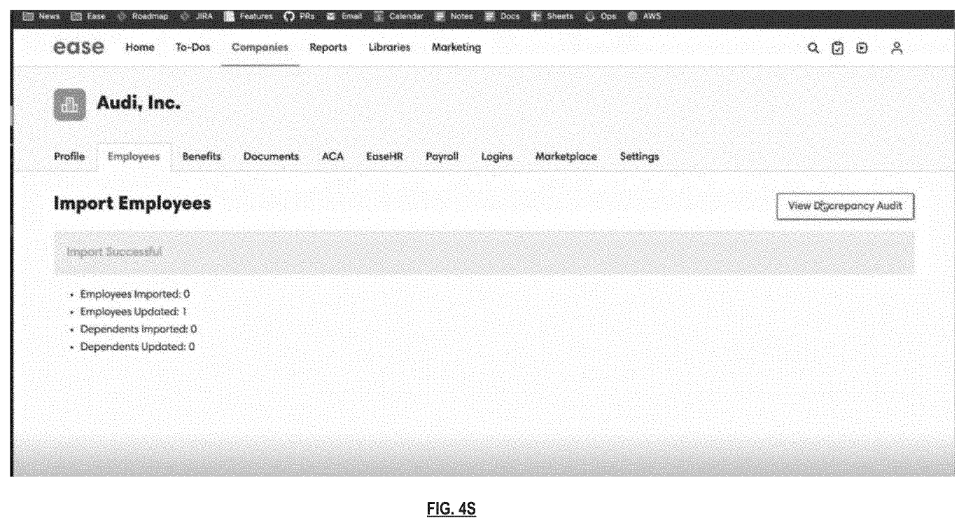

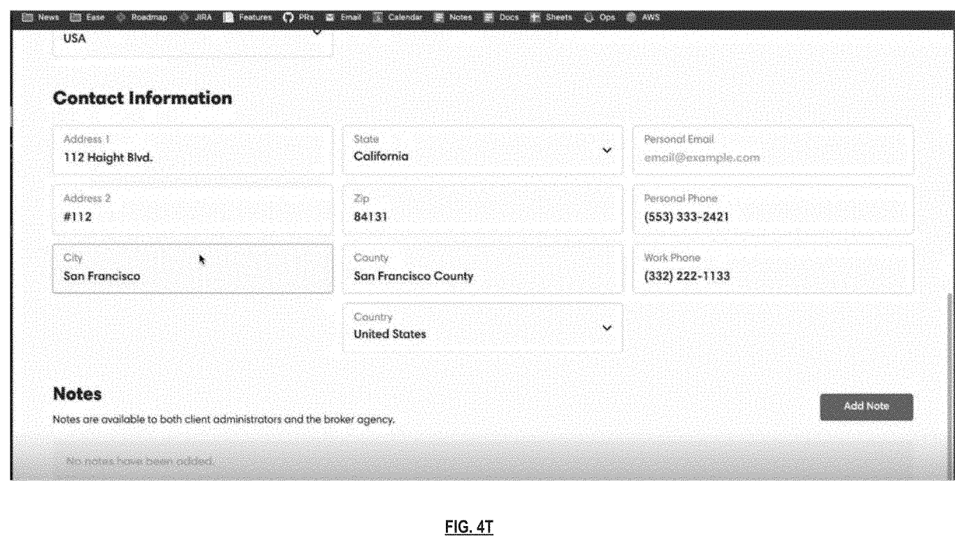

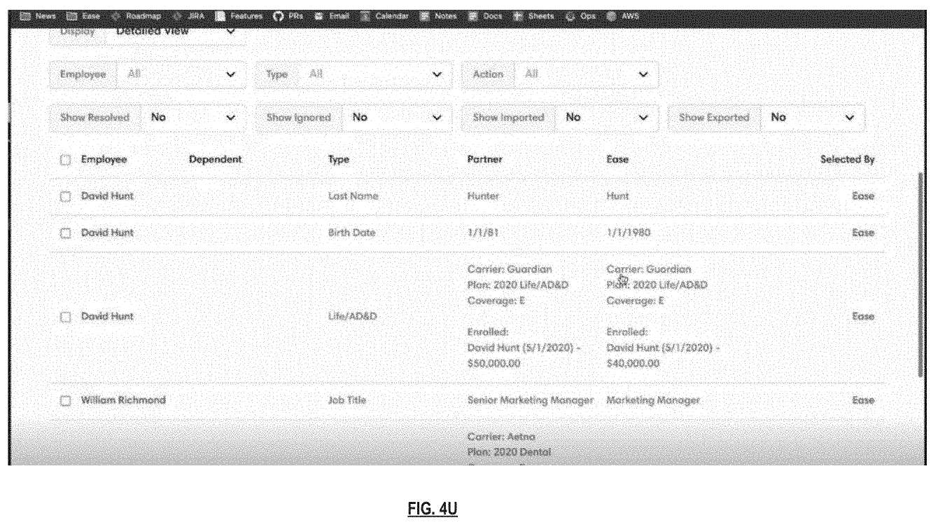

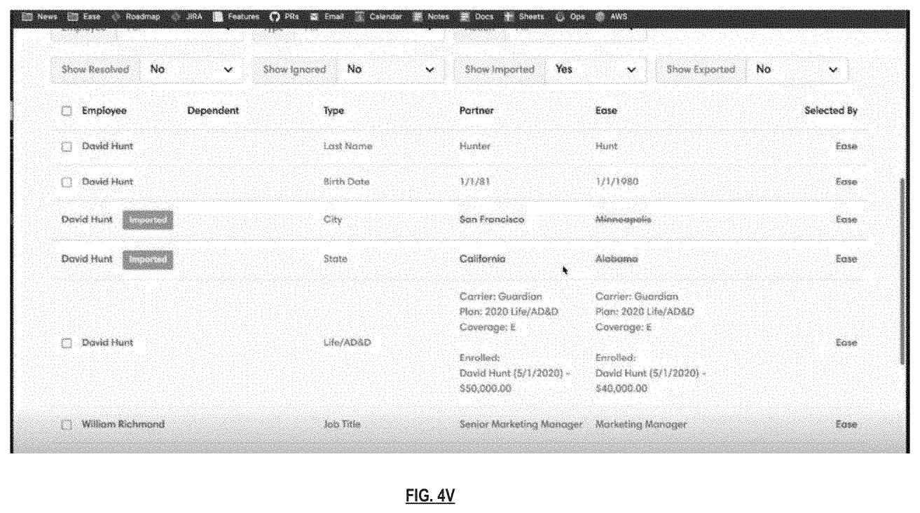

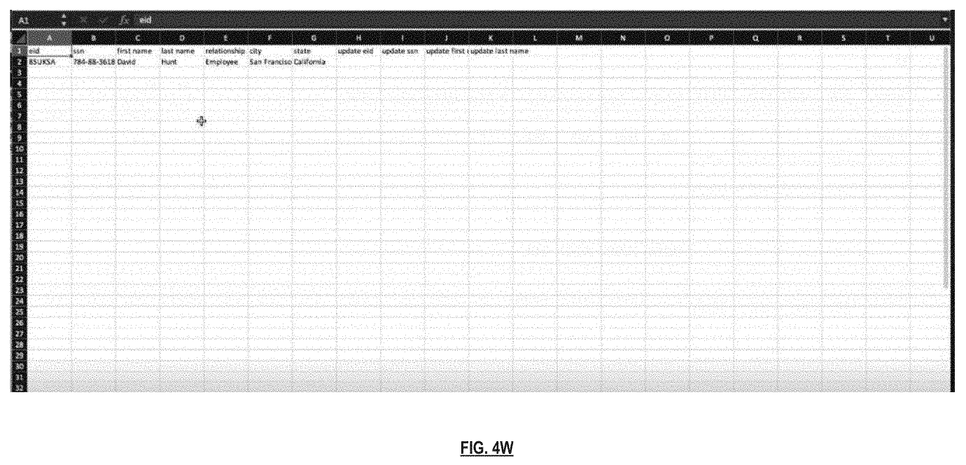

FIGS. 4A-4W are screen displays of an interactive interface in accordance with various embodiments. In particular various screen displays are presented that are examples of displays and user interactions with interactive interface 275 generated by the discrepancy audit system 100. In the example shown in FIG. 4A, the discrepancy audit system 100 is referred to as “ease” and is accessed via a user in reference to a partner “Audi, Inc.” that is hypothetical and not meant to represent any actual company or entity. The user can elect to create an audit of benefit records imported from partners in any of variety of different formats such as through an API or files such as CSV or other formats that may be delivered to the discrepancy audit system 100, by clicking “create audit”.

In FIG. 4B, a file “audi-audit.csv” is selected, a portion of whose content is shown in FIG. 4C by user selection to view the file. Clicking on “start import” in FIG. 4B starts the import of the partner benefit records. In FIG. 4D, the partner benefit records have been imported and several warnings have been generated. The user can elect to go back and correct these records in the file or can proceed with either downloading or viewing the audit based on user selection.

In FIG. 4E, the user has elected to view the discrepancy audit that results from the comparison of the partner benefits records that were imported to corresponding system benefits records from the ease system. In this example, the audit found 10 discrepancies from 4 employees and 1 dependent. The date of the audit is noted because things can change after this time. For example, an employee's, status, benefits, address, name, etc. may change with time so this audit data references only this snapshot in time.

Scrolling down via the interactive interface results in FIG. 4F presents a detailed view where several instances of discrepancies are noted between the partner benefits records in the “Partner” column and system benefits records in the “Ease” column. The type of each discrepancy and the employee name are also indicated in each instance. An employee view that shows a summary of discrepancies by employee is shown in FIG. 4G. As shown in FIG. 4H, a detailed view can also be selected to show discrepancies for benefits records associated with a selected employee “David Hunt”.

As previously discussed, the interactive interface 275 allows the user to select instances that are correct. In the example shown in FIG. 4I, the user has clicked on “Hunt” in the system column because “Hunt” and not “Hunter” is actually correct. Selecting the system value visually indicates this instance is correct by changing the text color from black to green. The system automatically, and without further user interaction, indicates that the value “Hunter” in the partner column is incorrect by crossing through the text and changing the text color to red. In this fashion, the user can scroll through the various discrepancies and manually select the values that are correct—with the system automatically flagging the incorrect values.

In FIG. 4J, the user has opened an “Actions” menu where “Run rules” can be selected to automatically determine whether particular instances of the systems benefits records or the partner benefits records appear to be correct or incorrect, based on the application of a set of rules. The results are shown in FIG. 4K. The set of rules have determined that a subset of system values for last name, birth date, city and state are correct, while another subset partner values for Life/AD&D are correct. The correct/incorrect indications have been automatically made in a similar mode to the manual indications of FIG. 4I. In other example however, a different form of indications can be used for rules-based versus manual-based indications. In addition, the user can still reverse any of these determinations by selecting one or more truly correct values that were labeled as incorrect based on the application of the set of rules. It should be further noted that the rules were unable to determine which job title was correct for David Hunt, forcing a manual selection to be made for this subset of the discrepancies as shown in FIG. 4L.

FIG. 4M presents a different case where the user elects to resolve the correct city and state for David Hunt by electing to view more detailed system information shown in in FIG. 4N. The county and zip codes provide clues to the user that the city and state information stored by the system are incorrect, and the user manually selects the correct partner value as shown in FIG. 4O. After these two corrections are indicated, the user has elected to import these updates to the system as shown in FIG. 4P as corrected systems benefits data. FIG. 4Q presents a summary indicating that records relating to one employee are being updated. The user elects to finish the import as shown in FIG. 4R and the success of the import is indicated by the interactive interface in FIG. 4S.

The detailed system information shown in FIG. 4T shows that David Hunt's system benefits records for city and state have been corrected. A further audit shown in FIG. 4U no longer shows a discrepancy for David Hunt's city and state records. A user election to show the imported data, however, indicates the imported corrections as shown in FIG. 4V. The import filed of corrections can be viewed as shown in FIG. 4W. The discrepancy audit system 100 can mark these imported values as previously corrected.

As previously noted, any corrections to corrections to the partner benefit records can be proactively exported to the partner database storage system 140 under user control via the interactive interface. In other embodiments, automatic file transfers or API calls can be employed to apply corrected partner benefits data to the partner database storage system 140, on a scheduled basis or a real time basis. This information can then automatically be updated to the right partner once this audit is complete.

The discrepancy audit system 100 presents an interactive interface 275 capable of handling very large audits in a quick and efficient manner. In particular, the discrepancy audit system 100 improves the technology of automated audit processing systems by automatically presenting discrepancies between system and partner records, providing a fast and efficient method for selecting which values are correct, and providing automated methodologies for updating benefits records without, or with minimal, user interaction.

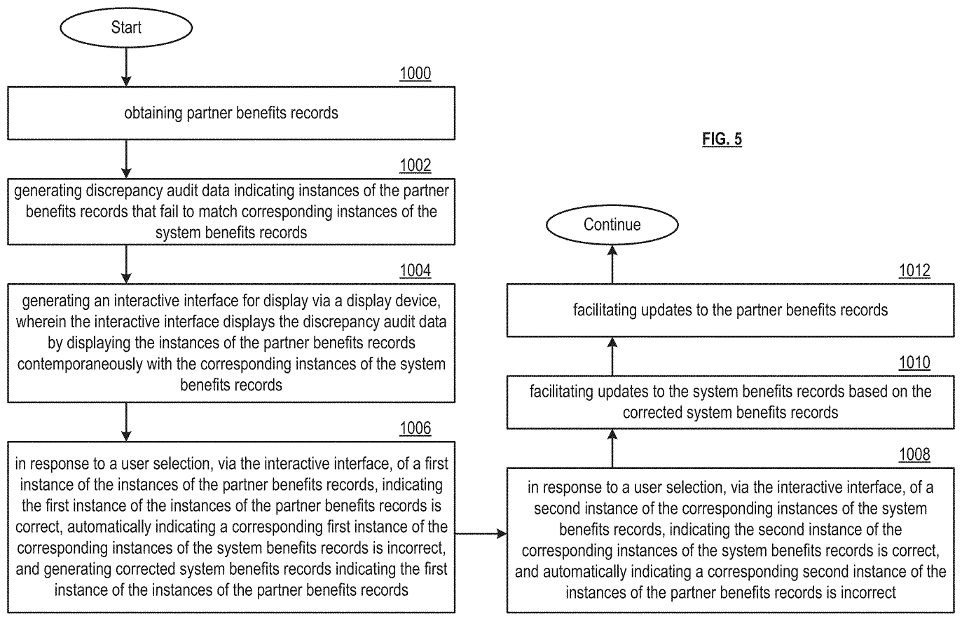

FIG. 5 is a flowchart representation of a method in accordance with various embodiments. In particular, a method is presented for use in conjunction with one or more functions and features previously described. Step 1000 includes obtaining partner benefits records. Step 1002 includes comparing the partner benefits records to system benefits records. Step 1004 includes generating discrepancy audit data indicating instances of the partner benefits records that fail to match corresponding instances of the system benefits records. Step 1006 includes generating an interactive interface for display via a display device, wherein the interactive interface displays the discrepancy audit data by displaying the instances of the partner benefits records contemporaneously with the corresponding instances of the system benefits records. Step 1008 includes indicating, in response to a user selection via the interactive interface, of a first instance of the instances of the partner benefits records, the first instance of the instances of the partner benefits records is correct, and automatically indicating a corresponding first instance of the corresponding instances of the system benefits records is incorrect, and generating corrected system benefits records indicating the first instance of the instances of the partner benefits records. Step 1010 includes indicating, in response to a user selection via the interactive interface, of a second instance of the corresponding instances of the system benefits records, the second instance of the corresponding instances of the system benefits records is correct, and automatically indicating a corresponding second instance of the instances of the partner benefits records is incorrect. Step 1012 includes facilitating updates to the system benefits records based on the corrected system benefits records. Step 1014 includes facilitating updates to the partner benefits records.

In various embodiments, the system benefits records are received from a system benefits database and facilitating updates to the system benefits records includes importing the corrected system benefits records to the system benefits database. The partner benefits records can be received from a partner benefits database and facilitating the updates to the partner benefits records can be based on the system benefits database. In the alternative, facilitating updates to the partner benefits records can include generating corrected partner benefits and exporting corrected partner benefits records to the partner benefits database.

In various embodiments, the method further includes: processing the instances of the partner benefits records and the corresponding instances of the system benefits records based on a set of rules; indicating, via the interactive interface and based on the processing, a first subset of the instances of the partner benefits records is correct and a corresponding first subset of the corresponding instances of the system benefits records is incorrect; and indicating, via the interactive interface and based on the processing, a second subset of the instances of the partner benefits records is incorrect and a corresponding second subset of the corresponding instances of the system benefits records is correct. In addition, when the first instance of the instances of the partner benefits records is a member of the second subset, the user selection, via the interactive interface, of the first instance of the instances of the partner benefits records reverses an indication, based on the processing, that the first instance of the instances of the partner benefits records is incorrect. Further, when the second instance of the corresponding instances of the system benefits records is a member of the first subset, the user selection, via the interactive interface, of the second instance of the corresponding instances of the system benefits records reverses an indication, based on the processing, that the second instance of the corresponding instances of the system benefits records is incorrect.

The method can further include indicating, via the interactive interface and based on the processing, a third subset of the instances of the partner benefits records and a corresponding third subset of the corresponding instances of the system benefits records cannot be determined to be either correct or incorrect.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provides an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributedly located (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims. Further, the boundaries of these functional building blocks have been arbitrarily defined for convenience of description. Alternate boundaries could be defined as long as the certain significant functions are appropriately performed. Similarly, flow diagram blocks may also have been arbitrarily defined herein to illustrate certain significant functionality.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, a quantum register or other quantum memory and/or any other device that stores data in a non-transitory manner. Furthermore, the memory device may be in a form of a solid-state memory, a hard drive memory or other disk storage, cloud memory, thumb drive, server memory, computing device memory, and/or other non-transitory medium for storing data. The storage of data includes temporary storage (i.e., data is lost when power is removed from the memory element) and/or persistent storage (i.e., data is retained when power is removed from the memory element). As used herein, a transitory medium shall mean one or more of: (a) a wired or wireless medium for the transportation of data as a signal from one computing device to another computing device for temporary storage or persistent storage; (b) a wired or wireless medium for the transportation of data as a signal within a computing device from one element of the computing device to another element of the computing device for temporary storage or persistent storage; (c) a wired or wireless medium for the transportation of data as a signal from one computing device to another computing device for processing the data by the other computing device; and (d) a wired or wireless medium for the transportation of data as a signal within a computing device from one element of the computing device to another element of the computing device for processing the data by the other element of the computing device. As may be used herein, a non-transitory computer readable memory is substantially equivalent to a computer readable memory. A non-transitory computer readable memory can also be referred to as a non-transitory computer readable storage medium.

While particular combinations of various functions and features of the one or more embodiments have been expressly described herein, other combinations of these features and functions are likewise possible. The present disclosure is not limited by the particular examples disclosed herein and expressly incorporates these other combinations.

What is claimed is:

1. A discrepancy audit system, comprising:
at least one processing system that includes a processor; and
at least one memory that stores executable instructions that, when executed by the at least one processing system, facilitate performance of operations comprising:
obtaining partner benefits records;
comparing the partner benefits records to system benefits records;
generating discrepancy audit data indicating instances of the partner benefits records that fail to match corresponding instances of the system benefits records;
generating an interactive interface for display via a display device, wherein the interactive interface displays the discrepancy audit data by displaying the instances of the partner benefits records contemporaneously with the corresponding instances of the system benefits records;
in response to a user selection via the interactive interface of a first instance of the instances of the partner benefits records, indicating the first instance of the instances of the partner benefits records is correct, automatically indicating a corresponding first instance of the corresponding instances of the system benefits records is incorrect, and generating corrected system benefits records indicating the first instance of the instances of the partner benefits records;
in response to a user selection via the interactive interface of a second instance of the corresponding instances of the system benefits records, indicating the second instance of the corresponding instances of the system benefits records is correct, and automatically indicating a corresponding second instance of the instances of the partner benefits records is incorrect;
facilitating updates to the system benefits records based on the corrected system benefits records; and
facilitating updates to the partner benefits records.

2. The discrepancy audit system of claim 1, wherein indicating the first instance of the instances of the partner benefits records is correct includes a first visual indication via the interactive interface, wherein automatically indicating the corresponding first instance of the instances of the partner benefits records is incorrect includes a second visual indication via the interactive interface, wherein indicating the second instance of the corresponding instances of the system benefits records is correct includes a third visual indication via the interactive interface, and wherein automatically indicating the corresponding second instance of the instances of the partner benefits records is incorrect includes a fourth visual indication via the interactive interface.

3. The discrepancy audit system of claim 1, wherein the system benefits records are received from a system benefits database and wherein facilitating updates to the system benefits records includes importing the corrected system benefits records to the system benefits database.

4. The discrepancy audit system of claim 3, wherein the partner benefits records are received from a partner benefits database and wherein facilitating the updates to the partner benefits records is based on the system benefits database.

5. The discrepancy audit system of claim 1, wherein the partner benefits records are received from a partner benefits database and wherein facilitating updates to the partner benefits records includes generating corrected partner benefits and exporting corrected partner benefits records to the partner benefits database.

6. The discrepancy audit system of claim 1, wherein obtaining partner benefits records includes importing a partner benefits record file, wherein the operations further comprise:
processing the partner benefits record file based on a plurality of warning conditions;
generating one or more warning notifications in response to the processing; and
presenting the one or more warning notifications for display via the interactive interface.

7. The discrepancy audit system of claim 1, wherein the operations further comprise:
responding to a user selection to present details corresponding to a selected one of the instances of the partner benefits records by presenting for display via the interactive interface the details corresponding to the selected one of the instances of the partner benefits records.

8. The discrepancy audit system of claim 1, wherein the operations further comprise:
responding to a user selection to present details corresponding to a selected one of the corresponding instances of the system benefits records by presenting for display via the interactive interface the details corresponding to the selected one of the corresponding instances of the system benefits records.

9. The discrepancy audit system of claim 1, wherein the operations further comprise:
processing the instances of the partner benefits records and the corresponding instances of the system benefits records based on a set of rules;
indicating, via the interactive interface and based on the processing, a first subset of the instances of the partner benefits records is correct and a corresponding first subset of the corresponding instances of the system benefits records is incorrect; and
indicating, via the interactive interface and based on the processing, a second subset of the instances of the partner benefits records is incorrect and a corresponding second subset of the corresponding instances of the system benefits records is correct.

10. The discrepancy audit system of claim 9, wherein, when the first instance of the instances of the partner benefits records is a member of the second subset, the user selection, via the interactive interface, of the first instance of the instances of the partner benefits records reverses an indication, based on the processing, that the first instance of the instances of the partner benefits records is incorrect.

11. The discrepancy audit system of claim 9, wherein, when the second instance of the corresponding instances of the system benefits records is a member of the first subset, the user selection, via the interactive interface, of the second instance of the corresponding instances of the system benefits records reverses an indication, based on the processing, that the second instance of the corresponding instances of the system benefits records is incorrect.

12. The discrepancy audit system of claim 9, wherein the operations further comprise:
indicating, via the interactive interface and based on the processing, a third subset of the instances of the partner benefits records and a corresponding third subset of the corresponding instances of the system benefits records cannot be determined to be either correct or incorrect.

13. The discrepancy audit system of claim 1, wherein the discrepancy audit data further includes at least one of: an audit time, an audit date, a number of the instances of the partner benefits records that fail to match the corresponding instances of the system benefits records, a number of different employees reflected in the instances of the partner benefits records that fail to match the corresponding instances of the system benefits records, or a number of dependents reflected in the instances of the partner benefits records that fail to match the corresponding instances of the system benefits records.

14. A method for execution via at least one processing system including a processing circuit, the method comprising:
obtaining partner benefits records;
comparing the partner benefits records to system benefits records;
generating discrepancy audit data indicating instances of the partner benefits records that fail to match corresponding instances of the system benefits records;
generating an interactive interface for display via a display device, wherein the interactive interface displays the discrepancy audit data by displaying the instances of the partner benefits records contemporaneously with the corresponding instances of the system benefits records;
in response to a user selection via the interactive interface of a first instance of the instances of the partner benefits records, indicating the first instance of the instances of the partner benefits records is correct, automatically indicating a corresponding first instance of the corresponding instances of the system benefits records is incorrect, and generating corrected system benefits records indicating the first instance of the instances of the partner benefits records;
in response to a user selection via the interactive interface of a second instance of the corresponding instances of the system benefits records, indicating the second instance of the corresponding instances of the system benefits records is correct, and automatically indicating a corresponding second instance of the instances of the partner benefits records is incorrect;
facilitating updates to the system benefits records based on the corrected system benefits records; and
facilitating updates to the partner benefits records.

15. The method of claim 14, wherein the system benefits records are received from a system benefits database and wherein facilitating updates to the system benefits records includes importing the corrected system benefits records to the system benefits database.

16. The method of claim 15, wherein the partner benefits records are received from a partner benefits database and wherein facilitating the updates to the partner benefits records is based on the system benefits database.

17. The method of claim 14, wherein the partner benefits records are received from a partner benefits database and wherein facilitating updates to the partner benefits records includes generating corrected partner benefits and exporting corrected partner benefits records to the partner benefits database.

18. The method of claim 14, further comprising:
processing the instances of the partner benefits records and the corresponding instances of the system benefits records based on a set of rules;
indicating, via the interactive interface and based on the processing, a first subset of the instances of the partner benefits records is correct and a corresponding first subset of the corresponding instances of the system benefits records is incorrect; and
indicating, via the interactive interface and based on the processing, a second subset of the instances of the partner benefits records is incorrect and a corresponding second subset of the corresponding instances of the system benefits records is correct.

19. The method of claim 18, wherein, when the first instance of the instances of the partner benefits records is a member of the second subset, the user selection, via the interactive interface, of the first instance of the instances of the partner benefits records reverses an indication, based on the processing, that the first instance of the instances of the partner benefits records is incorrect; and
wherein, when the second instance of the corresponding instances of the system benefits records is a member of the first subset, the user selection, via the interactive interface, of the second instance of the corresponding instances of the system benefits records reverses an indication, based on the processing, that the second instance of the corresponding instances of the system benefits records is incorrect.

20. The method of claim 18, further comprising:
indicating, via the interactive interface and based on the processing, a third subset of the instances of the partner benefits records and a corresponding third subset of the corresponding instances of the system benefits records cannot be determined to be either correct or incorrect.
